(19) United States
(12) Patent Application Publication
(10) Pub. No.: US 2023/0140545 A1
(43) Pub. Date: May 4, 2023

(54) GAS SENSOR, METHOD OF PRODUCING GAS SENSOR AND GAS MEASURING APPARATUS

(71) Applicant: TDK CORPORATION, Tokyo (JP)

(72) Inventors: Mayumi TAKAHASHI, Tokyo (JP); Kotaro TERAO, Tokyo (JP); Raitaro MASAOKA, Tokyo (JP)

(73) Assignee: TDK CORPORATION, Tokyo (JP)

(21) Appl. No.: 17/978,429

(22) Filed: Nov. 1, 2022

Related U.S. Application Data
(60) Provisional application No. 63/275,627, filed on Nov. 4, 2021.

(51) Int. Cl. G01N 27/60; G01N 33/00

(52) U.S. Cl. CPC G01N 27/60; G01N 33/0027

(57) ABSTRACT

A gas sensor includes a quartz crystal unit and a sensitive layer formed on the quartz crystal unit and configured to adsorb a gas to be detected, wherein the sensitive layer is porous, and has a particle skeleton made of a plurality of conductive polymer particles and a polyelectrolyte that is at least partially disposed between the adjacent conductive polymer particles.

### CROSS-REFERENCES TO RELATED APPLICATIONS

This application relies for priority upon U.S. Provisional Application No. 63/275.627 filed on Nov. 4, 2021 the entire content of which is hereby incorporated herein by reference for all purposes as if fully set forth herein.

### BACKGROUND

The present disclosure relates to a gas sensor, a method of producing a gas sensor and a gas measuring apparatus including a gas sensor.

A gas phase sample detection technology is important in both industrial and consumer market sectors. Gas sensors that detect the types and/or concentrations of gas phase samples are used in many fields and for various applications. For example, gas sensors are used to detect the types and/or concentrations of toxic gases and flammable gases. In addition, in a vacuum deposition process, gas sensors are used to detect the vapor concentration of materials in order to control the film thickness. In addition, gas sensors have also been used as humidity sensors for detecting the amount of water vapor. In recent years, gas sensors have also been used to detect volatile organic gases that cause sick house syndrome. In addition, gas sensors are used to detect diseases from exhaled breath.

In the related art, there are a plurality of gas sensor sensing methods such as resistance type, capacitance type, optical type, and mass type methods. A mass-type gas sensor uses a quartz crystal microbalance (QCM) method in which the mass of molecules is measured using oscillation of a quartz crystal unit and has high sensitivity. The mass-type gas sensor can be produced by a method of applying a resin composition onto a quartz crystal unit to form a polymer film that adsorbs a gas to be detected. Therefore, the gas sensor using a mass type sensing method can be produced more easily than, for example, one using a resistance type sensing method using a heating mechanism for forming an, oxide semiconductor for production.

A gas sensor including a quartz crystal unit and a polymer film formed on the quartz crystal unit and configured to adsorb a gas to be detected is described in, for example, Patent Document 1. Patent Document 1 describes an odor sensor including a physical adsorption film that adsorbs an odor substance and a quartz crystal unit sensor (QCM sensor), in which the physical adsorption film includes a conductive polymer and a dopant that modifies substance properties of the conductive polymer. In addition, Patent 1 discloses polyaniline and its derivatives as conductive polymers, and discloses polyacrylic acid or polystyrene sulfonic acid as a dopant. Patent Document 1 describes that a film solution obtained by mixing a polyaniline solution and a dopant solution is applied onto the surface of the QCM sensor, and dried to obtain a sensor element.

In addition, Patent Document 2 discloses, as a gas sensing material for a gas sensor apparatus, an ink material including nanoparticles (for example, metal oxide nanoparticles) and an organic material additive that can form an interconnection network of molecules (for example, to improve the porosity of synthesized nanoparticles). Patent Document 2 describes printing of an ink material on a gas sensor.

### Patent Documents

[Patent Document 1] U.S. Pat. No. 11073491

[Patent Document 2] U.S. Patent Application Publication No. 2017/0052161

### SUMMARY

A gas sensor including a quartz crystal unit and a polymer film that adsorbs a gas to be detected is required to have higher sensitivity and a wider range of a detectable gas concentration. The sensitivity of a gas sensor including a quartz crystal unit and a polymer film is greatly related to the material of the polymer film which is a sensitive film and a contact area between the polymer film and the gas to be detected.

As a method of increasing the sensitivity of a gas sensor, there is a method of dispersing a dopant in a polymer film to form a polymer film that easily adsorbs a gas. A polymer film in which a dopant is dispersed can be easily formed using a method in which a resin composition obtained by dissolving a resin and a dopant in a solvent is applied to a surface to be formed on the quartz crystal unit and dried. However, sufficient sensitivity is not obtained even with a gas sensor using a polymer film in which a dopant is dispersed. In addition, in a gas sensor using a polymer film in which a dopant is dispersed, in order to increase the sensitivity and widen a detectable gas concentration range, it is necessary to sufficiently widen the installation area of the polymer film. Therefore, it is difficult to reduce the size of a gas sensor using a polymer film in which a dopant is dispersed.

It is desirable to provide a gas sensor having a wide detectable gas concentration range with high sensitivity and a method of producing the same.

In addition, it is desirable to provide a gas measuring apparatus including a gas sensor having a wide detectable gas concentration range with high sensitivity.

The following means are provided.
[1] A gas sensor including a quartz crystal unit and a sensitive layer formed on the quartz crystal unit and configured to adsorb a gas to be detected,
   wherein the sensitive layer is porous, and has a particle skeleton made of a plurality of conductive polymer particles and a polyelectrolyte that is at least partially disposed between the adjacent conductive polymer particles.
[2] The gas sensor according to [1], wherein the sensitive layer has a surface porosity of 15% to 30%
[3] The gas sensor according to [1], wherein the sensitive layer has a density of 0.03 g/cm³ to 0.1 g/cm³.
[4] The gas sensor according to [1], wherein the conductive polymer particles contain an emeraldine salt of polyaniline represented by the following Formula (1):

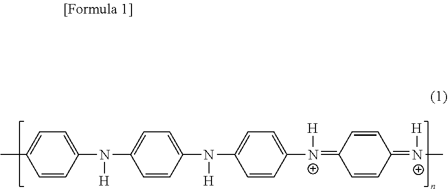

(in general Formula (1), n is a degree of polymerization).
[5] The gas sensor according to [4], wherein the polyelectrolyte contains any one, two or more of polystyrene sulfonic acid, polyacrylic acid, and, polyvinyl phosphonic acid.
[6] The gas sensor according to [5],
   wherein a buffer layer is disposed between the quartz crystal unit and the sensitive layer and in contact with the sensitive layer, and
   wherein an electrostatic adsorption layer containing any one, two, or more selected from among polystyrene sulfonic acid, polyacrylic acid, and polyvinyl phosphonic acid is formed on the surface of the buffer layer in contact with the sensitive layer.
[7] The gas sensor according to [1],
   wherein the quartz crystal unit includes a crystal plate and an electrode made of a metal film provided on the crystal plate,
   wherein the sensitive layer is laminated on the electrode with a buffer layer therebetween,
   wherein the electrode and the conductive polymer particles have a positive charge, and
   wherein the polyelectrolyte, the surface of the buffer layer in contact with the electrode, and the surface of the buffer layer in contact with the sensitive layer have a negative charge.
[8] A method of producing a gas sensor, including
   a sensitive layer forming step in which a porous sensitive layer that adsorbs a gas to be detected is formed on a quartz crystal unit,
   wherein the sensitive layer forming step includes a first step in which a first solution in which conductive polymer particles are dispersed is applied to the surface of the quartz crystal unit on which the sensitive layer is formed and dried; and
   a second step in which a second solution in which a polyelectrolyte is dissolved is applied to the sensitive layer forming surface after the first step and dried.
[9] The method of producing a gas sensor according to [8], wherein the conductive polymer particles contain polyaniline represented by the following Formula (1):

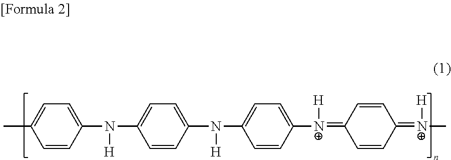

(in general Formula (1), n is a degree of polymerization).
[10] The method of producing a gas sensor according to [9], wherein the polyelectrolyte contains any one, two, or more selected from among polystyrene sulfonic acid, polyacrylic acid, and polyvinyl phosphonic acid.
[11] The method of producing a gas sensor according to [9], including
   a buffer layer forming step in which a buffer layer including an electrostatic adsorption layer containing any one, two, or more selected from among polystyrene sulfonic acid, polyacrylic acid, and polyvinyl phosphonic acid is formed on the sensitive layer forming surface before the sensitive layer forming step,
   wherein, in the sensitive layer forming step, the sensitive layer in contact with the electrostatic adsorption layer is formed.
[12] The method of producing a gas sensor according to [8],
   wherein the quartz crystal unit includes a crystal plate and an electrode made of a metal film provided on the crystal plate,
   wherein the method includes a buffer layer forming step in which, on the electrode, a buffer layer having a negative charge is formed on the surface in contact with the electrode and the surface in contact with the sensitive layer,
   wherein, in the first step, a first solution in which conductive polymer particles having a positive charge are dispersed is used, and
   wherein, in the second step, a second solution in which a polyelectrolyte having a negative charge is dissolved is used.
[13] The method of producing a gas sensor according to [8], wherein, in the sensitive layer forming step, a set of the first step and the second step is repeated a plurality of times.
[14] A gas measuring apparatus including the gas sensor according to [1].

A gas sensor of the present disclosure has a quartz crystal unit and a sensitive layer formed on the quartz crystal unit and configured to adsorb a gas to be detected, wherein the sensitive layer is porous and has a particle skeleton made of a plurality of conductive polymer particles and a polyelectrolyte that is at least partially disposed between the adjacent conductive polymer particles. Therefore, the sensitive layer of the gas sensor of the present disclosure has a larger contact area with the gas to be detected when the installation area is the same as compared with a non-porous sensitive film. As a result, the gas sensor of the present disclosure has high sensitivity and a wide detectable gas concentration range as compared with a gas sensor including a non-porous sensitive film. Therefore, the gas sensor of the present disclosure can be made small.

In the method of producing a gas sensor of the present disclosure, using a method of applying a first solution in which conductive polymer particles are dispersed onto the surface of the quartz crystal unit on which the sensitive layer is formed and performing drying and then applying a second solution in which a polyelectrolyte is dissolved and performing drying, a porous sensitive layer configured to adsorb a gas to be detected is formed. Therefore, according to the method of producing a gas sensor of the present disclosure, it is possible to produce the gas sensor of the present disclosure.

### BRIEF DESCRIPTION OF THE DRAWINGS

FIG. 1 is a plan view showing an example of a gas sensor according to the present embodiment.

FIG. 2 is a cross-sectional view of the gas sensor according to the present embodiment along the line A-A′ shown in FIG. 1.

FIG. 3 is an enlarged view showing an enlarged part of FIG. 2.

FIG. 4 is a flowchart for illustrating an example of a method of producing a gas sensor according to the present embodiment.

FIG. 5 is a step diagram for illustrating an example of a method of producing a gas sensor according to the present embodiment.

FIG. 6 shows schematic cross-sectional views for illustrating effects of the gas sensor according to the present embodiment.

FIG. 7 shows schematic cross-sectional views for illustrating an example of a gas sensor in the related art.

FIG. 8 is a schematic view showing a gas measuring apparatus of the present embodiment.

FIG. 9 is a microscopic image of a sensitive layer of a gas sensor 1.

FIG. 10 is a microscopic image of a sensitive film of a gas sensor of a comparative example.

### DETAILED DESCRIPTION

Hereinafter, a gas sensor, a method of producing a gas sensor and a gas measuring apparatus of the present disclosure will be described in detail with reference to the drawings.

### Gas Sensor

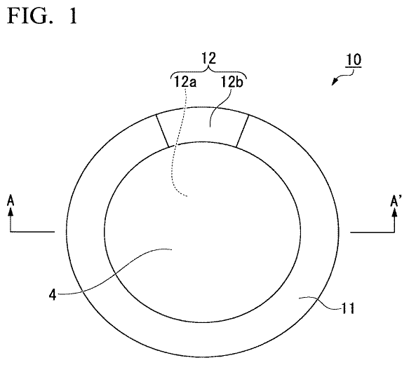

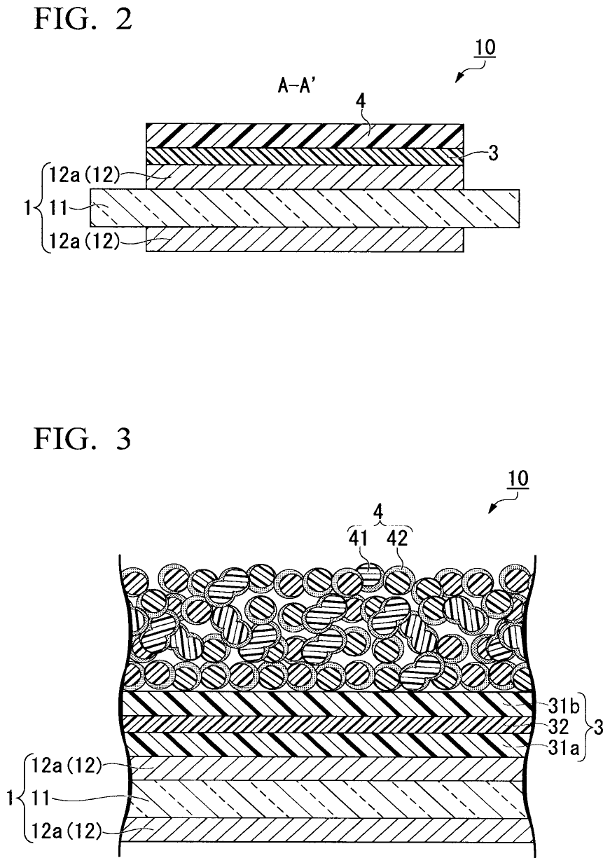

FIG. 1 is a plan view showing an example of a gas sensor according to the present embodiment. FIG. 2 is a cross-sectional view of the gas sensor according to the present embodiment along the line A-A′ shown in FIG. 1. FIG. 3 is an enlarged view showing an enlarged part of FIG. 2.

A gas sensor 10 of the present embodiment shown in FIG. 1 quantifies the concentration of a gas to be detected using a quartz crystal microbalance (QCM) method. As shown in FIG. 1, the gas sensor 10 of the present embodiment has a substantially circular shape in a plan view. As shown in FIG. 2, the gas sensor 10 has a sensitive layer 4 laminated on one electrode 12 between electrodes 12 provided on both surfaces of a quartz crystal unit 1 with a buffer layer 3 therebetween. The gas sensor 10 measures the oscillation vibration (frequency) of the quartz crystal unit 1 by two lead wires (not shown) electrically connected to the two electrodes 12 and is connected to a frequency measuring apparatus configured to detect the amount of change in the frequency. Known lead wires and frequency measuring apparatuses can be used.

(Quartz crystal unit)

As shown in FIG. 2, the quartz crystal unit 1 includes a crystal plate 11 and the electrodes 12 provided on both surfaces of the crystal plate 11. The fundamental frequency of the quartz crystal unit 1 changes in proportion to the change in the mass of the buffer layer 3 and the sensitive layer 4 disposed on one electrode 12. That is, when the sensitive layer 4 adsorbs a gas to be detected and the mass of the sensitive layer 4 increases, the fundamental frequency of the quartz crystal unit 1 decreases.

As the crystal plate 11, a known crystal plate can be used. In the gas sensor 10 of the present embodiment, a substantially circular crystal plate 11 in a plan view is used. The diameter of the crystal plate 11 may be, for example, 0.1 mm to 25 mm, and is preferably 0.5 mm to 10 mm. The thickness of the crystal plate 11 may be, for example, 50 μm to 500 μm , and is preferably 100 μm to 300 μm . The fundamental frequency when an electric field is applied to the electrode 12 of the quartz crystal unit 1 is determined according to the thickness of the crystal plate 11. When the thickness of the crystal plate 11 is thinner, the sensitivity is higher. On the other hand, when the thickness of the crystal plate 11 is thicker, the range of a detectable gas concentration is wider. Therefore, the thickness of the crystal plate 11 can be appropriately determined according to applications of the gas sensor 10 such as the concentration of the gas to be detected.

In the gas sensor 10 of the present embodiment, a case using a substantially circular crystal plate 11 in a plan view will be exemplified, but the shape of the crystal plate 11 is not limited to the substantially circular shape in a plan view, and can be appropriately determined according to applications of the gas sensor 10.

The electrodes 12 are made of metal films provided on both surfaces of the crystal plate 11. In a plan view, each electrode 12 has a circular central area 12a having the same center as the crystal plate 11 and a connection area 12b that extends from the edge of the central area 12a to the edge of the crystal plate 11.

The central area 12a of one electrode 12 is used as a sensitive layer forming surface on the quartz crystal unit 1 on which the sensitive layer 4 is formed. As shown in FIG. 1 and FIG. 2, the central area 12a of one electrode 12 is covered with the buffer layer 3 and the sensitive layer 4, which has substantially the same shape as the central area 12a in a plan view.

In addition, as shown in FIG. 1, the connection area 12b of the electrode 12 is exposed to the surface of the gas sensor 10. The connection area 12b is an area for connecting a lead wire (not shown) that electrically connects the gas sensor 10 to a frequency measuring apparatus. The connection areas 12b of the two electrodes 12 may be disposed at positions overlapping each other in a plan view or may be disposed at positions not overlapping each other in a plan view.

The electrode 12 is made of a metal film. As the material of the metal film, for example, gold, silver, copper, platinum or the like can be used. The electrode 12 preferably has a positive charge. The electrode 12 can be formed by a known method.

In the gas sensor 10 of the present embodiment, a case in which the electrode 12 has the circular central area 12a and the connection area 12b that extends from the edge of the central area 12a to the edge of the crystal plate 11 will be exemplified, but the planar shape and thickness of the electrode 12 are not particularly limited.

### (Sensitive Layer)

The sensitive layer 4 adsorbs a gas to be detected. The sensitive layer 4 is formed on the surface of the quartz crystal unit 1 on which the sensitive layer is formed. The sensitive layer 4 is porous, and as shown in FIG. 3, has a particle skeleton made of a plurality of conductive polymer particles 41 and a polyelectrolyte 42 that is at least partially disposed between the adjacent conductive polymer particles 41. The particle skeleton is formed by bonding the conductive polymer particles 41 to each other via the polyelectrolyte 42 in the plane direction and the lamination direction. Thereby, a large number of pores are formed in the sensitive layer 4.

The sensitive layer 4 has a surface porosity that is preferably 15% to 30%, and more preferably 18% to 28%. When the surface porosity is in a range of 15% to 30%, the contact area between the porous sensitive layer 4 and the gas to be detected is sufficiently wider than the contact area between the non-porous sensitive film and the gas. As a result, compared to a gas sensor including a non-porous sensitive film, the gas sensor 10 of the present embodiment has high sensitivity and a wide detectable gas concentration range when the installation areas of the sensitive layer 4 and the sensitive film are the same.

The density of the sensitive layer 4 is preferably 0.03 g/cm³ to 0.1 g/cm³ and more preferably 0.05 g/cm³ to 0.08 g/cm³. When the density of the sensitive layer 4 is in a range of 0.03 g/cm³ to 0.1 g/cm³, the contact area between the porous sensitive layer 4 and the gas to be detected is sufficiently larger than the contact area between the non-porous sensitive film and the gas and the amount of a gas that can be adsorbed is large. As a result, compared to a gas sensor including a non-porous sensitive film, the gas sensor 10 of the present embodiment has high sensitivity and a wide detectable gas concentration range when the installation areas of the sensitive layer 4 and the sensitive film are the same.

The thickness of the sensitive layer 4 may be, for example, 0.1 μm to 100 μm , and is preferably 1 μm to 50 μm, and can be appropriately determined according to the type and concentration of the gas to be detected. When the thickness of the sensitive layer 4 is 0.1 μm or more, this is preferable because the contact area between the porous sensitive layer 4 and the gas to be detected is sufficiently larger than the contact area between the non-porous sensitive film and the gas, and the amount of a gas that can be adsorbed is large. When the thickness of the sensitive layer 4 is 100 μm or less, this is preferable because the sensitive layer 4 can be efficiently formed.

The conductive polymer particles 41 forming the sensitive layer 4 may have a substantially spherical shape, an amorphous shape, or a shape formed by aggregating a plurality of particles.

In the gas sensor 10 of the present embodiment, it is preferable that the surfaces of the conductive polymer particles 41 and the polyelectrolyte 42 be attracted by electrostatic attraction. That is, the sensitive layer 4 is preferably formed by electrostatically aggregating the conductive polymer particles 41 and the polyelectrolyte 42. In this case, the surface of the conductive polymer particles 41 may have a positive charge, the polyelectrolyte 42 may have a negative charge, the surface of the conductive polymer particles 41 may have a negative charge, and the polyelectrolyte 42 may have a positive charge. Whether the surface charge is positive or negative can be confirmed by a known potential measurement method.

When the surface of the conductive polymer particles 41 has a positive charge and the polyelectrolyte 42 has a negative charge, a gas having a negative charge tends to be adsorbed on the surface of the conductive polymer particles 41, and a gas having a positive charge tends to be adsorbed on the polyelectrolyte 42. In addition, when the surface of the conductive polymer particles 41 has a negative charge and the polyelectrolyte 42 has a positive charge, a gas having a positive charge tends to be adsorbed on the surface of the conductive polymer particles 41 and a gas having a negative charge tends to be adsorbed on the polyelectrolyte 42. Accordingly, when the conductive polymer particles 41 and the polyelectrolyte 42 are electrostatically aggregated, favorable sensitivity is obtained when the gas to be detected is a gas having a positive charge or a gas having a negative charge.

When the surface of the conductive polymer particles 41 has a positive charge and the polyelectrolyte 42 has a negative charge, the polyelectrolyte 42 is adsorbed to the surface of the conductive polymer particles 41 by electrostatic attraction, and at least a part of the surface of the conductive polymer particles 41 is negatively charged. The conductive polymer particles 41 to which the polyelectrolyte 42 is adsorbed are attracted to other conductive polymer particles 41 by electrostatic attraction. Thereby, the conductive polymer particles 41 and the polyelectrolyte 42 are electrostatically aggregated to form the sensitive layer 4.

When the surface of the conductive polymer particles 41 has a positive charge and the polyelectrolyte 42 has a negative charge, as the material of the conductive polymer particles 41, for example, an emeraldine salt of polyaniline represented by the following Formula (1), polypyrrole, polyacetylene, polythiophene or the like can be used. Among these, the conductive polymer particles 41 preferably contain an emeraldine salt of polyaniline represented by the following Formula (1) because a sufficient charge density can be obtained.

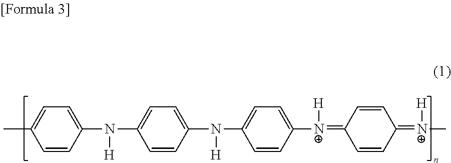

(in general Formula (1), n is a degree'of polymerization)

Polyaniline (PANI) has four structures of leucoemeraldine, an emeraldine base, pernigraniline, and an emeraldine salt represented by Formula (1). Among these, leucoemeraldine, emeraldine base, and pernigraniline have insulation properties, and only the emeraldine salt of polyaniline represented by Formula (1) has conductivity. The emeraldine salt of polyaniline represented by Formula (1) can be produced by a known method such as an electrolytic polymerization method.

The conductive polymer particles 41 containing the emeraldine salt of polyaniline represented by Formula (1) can be produced by a known method, for example, a suspension polymerization method. The particle size (D50) of the conductive polymer particles 41 may be, for example, 0.03 μm to 2 μm. The particle size of the conductive polymer particles 41 is a particle size of particles before aggregation predicted from the state of the aggregated particles when the conductive polymer particles 41 have a form in which a plurality of particles are aggregated.

As the conductive polymer particles 41 containing the emeraldine salt of polyaniline represented by Formula (1), commercially available conductive polymer particles may be used. For example, a polyaniline emeraldine salt (commercially available from Sigma-Aldrich), and a polyaniline emeraldine salt (commercially available from FUJIFILM Wako Pure Chemical Corporation) may be exemplified.

When the surface of the conductive polymer particles 41 has a positive charge and the polyelectrolyte 42 has a negative charge, as the material of the polyelectrolyte 42, for example, polystyrene sulfonic acid (PSS), polyacrylic acid, polyvinyl phosphonic acid or the like can be used. Among these, the polyelectrolyte 42 preferably contains any one, two or more selected from among polystyrene sulfonic acid, polyacrylic acid, and polyvinyl phosphoric acid. In, this case, since the electrostatic attraction between the polyelectrolyte 42 and the conductive polymer particles 41 is strong, it is possible to secure the strength of the sensitive layer 4. Moreover, since a strong negative charge having a sufficient charge density can be imparted to the surface of the conductive polymer particles 41 (that is, the surface of the sensitive layer 4), the conductive polymer particles 41 function as the highly sensitive layer 4 that easily adsorbs a gas.

The polyelectrolyte 42 more preferably contains polystyrene sulfonic acid and/or polyacrylic acid and most preferably contains polystyrene sulfonic acid.

Particularly, when the conductive polymer particles 41 contain an emeraldine salt of polyaniline represented by Formula (1) and the polyelectrollyte 42 contains polystyrene sulfonic acid, this is preferable because the polyelectrolyte 42 is adsorbed to the conductive polymer particles 41 to form a stable state, and the sensitive layer 4 with higher sensitivity that easily adsorbs a gas having a positive charge or a gas having a negative charge is formed.

### (Buffer Layer)

As shown in FIG. 2, the buffer layer 3 is disposed between the electrode 12 of the quartz crystal unit 1 and the sensitive layer 4 and in contact with the sensitive layer 4. The buffer layer 3 may be composed of only one layer or may be composed of a plurality of layers. When the buffer layer 3 is composed of a plurality of layers, for example, as shown in FIG. 3, it is preferable to have a laminated structure in which a first buffer layer 31a, a second buffer layer 32 and a third buffer layer 31b are laminated in order from the side of the quartz crystal unit 1. In the example shown in FIG. 3, a case in which one first buffer layer 31a and one second buffer layer 32 are provided will be exemplified, but a plurality of first buffer layers 31a and second buffer layers 32 may be alternately provided.

When the surface of the conductive polymer particles 41 on which the sensitive layer 4 is formed has a positive charge and the polyelectrolyte 42 has a negative charge, the surface of the buffer layer 3 in contact with the sensitive layer 4 preferably has a negative charge. In the example shown in FIG. 3, the surface of the third buffer layer 31b in contact with the sensitive layer 4 preferably has a negative charge. Thereby, the surface of the buffer layer 3 in contact with the sensitive layer 4 functions as an electrostatic adsorption layer for the sensitive layer 4.

When the surface in contact with the sensitive layer 4 has a negative charge, as the material of the buffer layer 3 (in the example shown, in FIG. 3, the third buffer layer 31b) forming the surface in contact with the sensitive layer 4, the same material that can be used when the polyelectrolyte 42 has a negative charge can be used. The surface of the buffer layer 3 in contact with the sensitive layer 4 and the polyelectrolyte 42 may be made of the same material or may be made of different materials.

When the surface of the buffer layer 3 in contact with the sensitive layer 4 has a negative charge, as the material of the buffer layer 3, it preferably contains any one, two or more selected from among polystyrene sulfonic acid, polyacrylic acid, and polyvinyl phosphonic acid, more preferably contains polystyrene sulfonic acid and/or polyacrylic acid, and most preferably contains polystyrene sulfonic acid because a sufficient charge density can be obtained and the function of the sensitive layer 4 as an electrostatic adsorption layer is improved.

When the surface of the conductive polymer particles 41 has a positive charge and the polyelectrolyte 42 has a negative charge, it is preferable that the first buffer layer 31a and the third buffer layer 31b have a negative charge and the second buffer layer 32 have a positive charge.

In this case, as the material of the first buffer layer 31a, the same material that can be used for the third buffer layer 31b can be used.

As the material of the second buffer layer 32, for example, polydiallyldimethylammonium chloride (PDDA), polydimethyldiglylammonium chloride (PDMDAA), polyallylamine hydrochloride, quaternized poly(vinylpyridine)hydrochloride or the like can be used. Among these, it is preferable to contain polydiallyldimethylammonium chloride because a sufficient charge density can be obtained.

When the first buffer layer 31a and the third buffer layer 31b have a negative charge and the second buffer layer 32 has a positive charge, the function of the sensitive layer 4 as an electrostatic adsorption layer is further improved. That is, the second buffer layer 32 is attracted to the surface of the first buffer layer 31a by electrostatic attraction and the surface is positively charged. The third buffer layer 31b is attracted to the attracted surface of the second buffer layer 32 by electrostatic attraction and the surface is negatively charged. Thereby, compared to then the buffer layer 3 is composed of only the first buffer layer 31a, the charge density on the surface of the buffer layer 3 increases and electrostatic attraction with which the sensitive layer 4 is attracted is strengthened. In addition, when a plurality of first buffer layers 31a and second buffer layers 32 are alternately provided, the electrostatic attraction with which the sensitive layer 4 is attracted can be further strengthened.

In addition, when the electrode 12 has a positive charge, this is preferable because the first buffer layer 31a of the buffer layer 3 shown in FIG. 3 has a negative charge, and thus the first buffer layer 31a can be attracted to the surface of the electrode 12 by electrostatic attraction.

### “Method of Producing Gas Sensor”

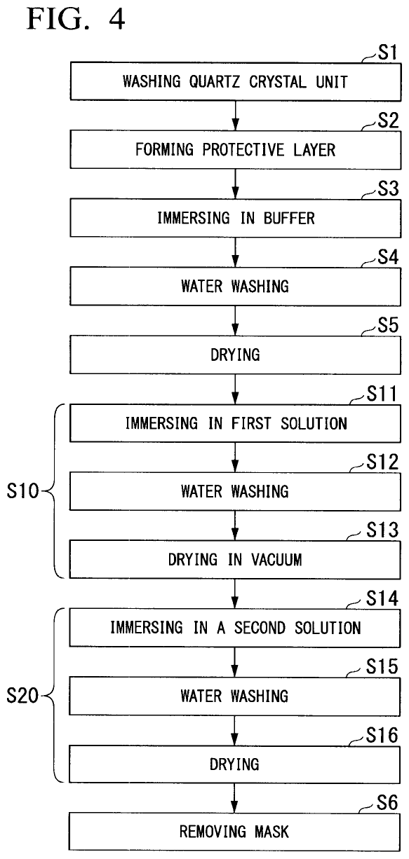

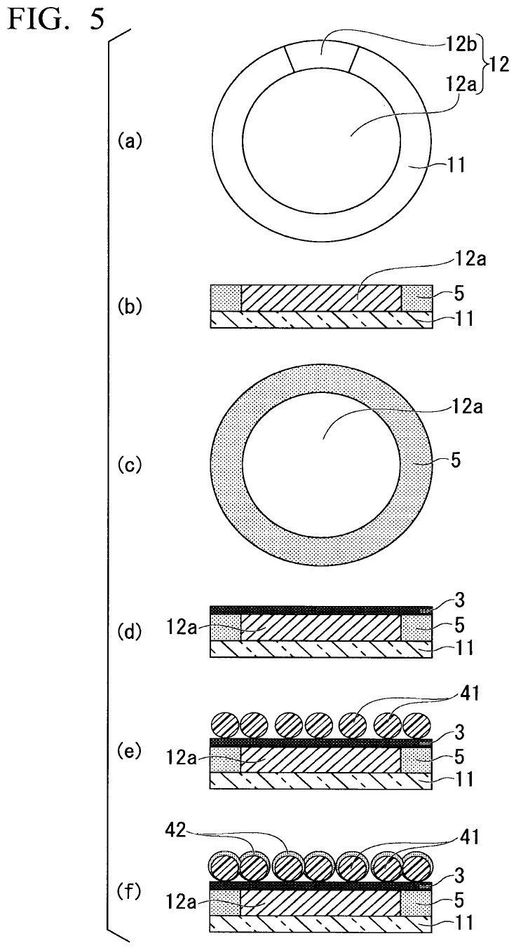

Next, a method of producing a gas sensor according to the present embodiment will be exemplified. FIG. 4 is a flowchart for illustrating an example of the method of producing a gas sensor according to the present embodiment. FIG. 5 is a step diagram for illustrating an example of the method of producing a gas sensor according to the present embodiment. FIG. 5(a) and FIG. 5(c) are plan views of a gas sensor during production. FIG. 5(b), and FIG. 5(d) to FIG. 5(f) are cross-sectional views of a gas sensor during production corresponding to the positions along the line A-A′ shown in FIG. 1.

In the method of producing a gas sensor according to the present embodiment, first, as shown in FIG. 5(a), the quartz crystal unit 1 including the crystal plate 11 and the electrodes 12 provided on both surfaces of the crystal plate 11 is prepared. Next, as shown in FIG. 4, both surfaces of the quartz crystal unit 1 are washed using a known method such as ultrasonic washing, ultraviolet light emission, an ozone treatment, and a plasma treatment (S1).

Next, a protective mask is formed on the surface of the quartz crystal unit 1 (S2). As shown in FIG. 5(b) and FIG. 5(c), a protective mask 5 is formed on one surface of the quartz crystal unit 1 to cover the surface of the quartz crystal unit 1 by exposing only the central area 12a (sensitive layer forming surface) of the electrode 12. The protective mask 5 is also formed on the entire other surface of the quartz crystal unit 1. As the protective mask 5, a material that is insoluble in a first solution to be described below and insoluble in water can be used, and for example, known materials such as a photocurable resin film such as a UV-curing resin film, a thermosetting resin film, a thermal release resin sheet, and a masking tape can be used.

### (Buffer Layer Forming Step)

Next, as shown in FIG. 5(d), a buffer layer forming step in which the buffer layer 3 is formed on the central area 12a (sensitive layer forming surface) of the electrode 12 in the quartz crystal unit 1 on which the protective mask 5 is formed is performed. In the buffer layer forming step, it is preferable to use an electrostatic adsorption composite method in which a surface charge is controlled and substances are laminated by electrostatic attraction.

First, a buffer solution is prepared by dissolving the material that forms the above buffer layer 3 in a solvent. The solvent can be appropriately determined according to the type of the material that forms the buffer layer 3, and for example, water, methanol, ethanol or the like can be used.

Next, as shown in FIG. 4, the quartz crystal unit 1 with the protective mask 5 formed is immersed in a buffer solution, and the buffer solution is applied (S3). Then, the quartz crystal unit 1 is removed from the buffer solution and washed with water (S4), and dried in, for example, a nitrogen atmosphere at 80° C. for 10 minutes (S5).

When the buffer layer 3 is formed of a plurality of layers, a buffer solution corresponding to the material of the buffer layer 3 is prepared for each layer, and the steps (S3) to (S5) in FIG. 4 are repeated for the number of laminations.

In the present embodiment as the sensitive layer 4, when a sensitive layer in which the surface of the conductive polymer particles 41 has a positive charge and the polyelectrolyte 42 has a negative charge is produced, the surface of the buffer layer 3 in contact with the sensitive layer 4 is formed using the buffer solution corresponding to the material having a negative charge described above, and thus the buffer layer 3 having an electrostatic adsorption layer can be formed.

In addition, when the electrode 12 has a positive charge, the surface of the buffer layer 3 in contact with the electrode 12 is formed using the buffer solution corresponding to the material having a negative charge described above, and thus the buffer layer 3 can be attracted to the surface of the electrode 12 by electrostatic attraction.

### (Sensitive Layer Forming Step)

Next, a sensitive layer forming step in which the porous sensitive layer 4 that adsorbs a gas to be detected is formed on the quartz crystal unit 1 with the buffer layer 3 formed is performed. Also in the sensitive layer forming step, it is preferable to use an electrostatic adsorption composite method.

In the present embodiment, as shown in FIG. 4, as the sensitive layer forming step, a first step S10 and a second step S20 are performed.

### (First Step)

First, a first solution in which the above conductive polymer particles 41 are dispersed in a solvent is prepared.

Any solvent in which the conductive polymer particles 41 are not dissolved may be used, and for example, N-methylpyrrolidone (NMP), dimethylformamide (DMF), dimethyl sulfoxide (DMSO), pyridine, butylamine, isophorone or the like can be used. When a solvent in which the conductive polymer particles 41 are not dissolved is used, the conductive polymer particles 41 can adhere to the sensitive layer forming surface of the quartz crystal unit 1 while the shape of the conductive polymer particles 41 is maintained.

As the conductive polymer particles 41 dispersed in a solvent, those having, for example, a particle size (D50) of 0.03 μm to 2 μm can be used. As necessary, the conductive polymer particles 41 that are classified using a filter, a swirling airflow-driven air classifier or the like so that they have a predetermined particle size and particle size distribution are used. The particle size (D50) of the conductive polymer particles 41 can be appropriately determined according to a desired density of the sensitive layer 4.

Next, as shown in FIG. 4, the quartz crystal unit 1 with the buffer layer 3 formed is immersed in a first solution, and the first solution is applied (S11). The immersion time for which the quartz crystal unit 1 is immersed in the first solution can be set to, for example, 10 seconds to 40 seconds, and can be appropriately determined according to a desired surface porosity of the sensitive layer 4. Then, the quartz crystal unit 1 is removed from the first solution and washed with rater (S12), and vacuum-dried, for example, at 80° C. for 10 minutes (S13).

In the present embodiment, instead of vacuum drying (S13), drying may be performed in an oxygen-free atmosphere such as a nitrogen gas atmosphere or an argon gas atmosphere. When drying after washing (S12) in the first, step S10 is performed in an oxygen-free atmosphere, and thus the conductive polymer particles 41 are an emeraldine salt of polyaniline represented by Formula (1), it is possible to prevent the conductive polymer particles 41 from being oxidized during drying. Therefore, it is possible to prevent the positive charge on the surface of the conductive polymer particles 41 from decreasing due to oxidation of the emeraldine salt of polyaniline represented by Formula (1).

When the above steps are performed, as shown in FIG. 5(e), the conductive polymer particles 41 adhere to the sensitive layer forming surface of the quartz crystal unit 1 with the buffer layer 3 therebetween.

### (Second Step)

First, a second solution in which the above polyelectrolyte 42 is dissolved in a solvent is prepared. Any solvent in which the polyelectrolyte 42 is dissolved may be used, and for example, water, methanol, ethanol or the like can be used.

Next, as shown in FIG. 4, the quartz crystal unit 1 to which the conductive polymer particles 41 adhere is immersed in a second solution, and the second solution is applied (S14). Then, the quartz crystal unit 1 is removed from the second solution and washed with water (S15), and dried, for example, in a nitrogen atmosphere at 80° C. for 10 minutes (S16). Thereby, as shown in FIG. 5(f), the polyelectrolyte 42 adheres to the conductive polymer particles 41 formed on the sensitive layer forming surface of the quartz crystal unit 1.

The first step S10 and the second step S20 shown in FIG. 4 may each be performed only once, and a set of the first step S10 and the second step S20 may be repeated a plurality of times. When a set of the first step S10 and the second step S20 are repeated a plurality of times, the conductive polymer particles 41 can be additionally laminated on the conductive polymer particles 41 to which the polyelectrolyte 42 adheres after the second step S20 is performed for the first time (refer to FIG. 5(f). Therefore, when a set of the first step S10 and the second step S20 are repeated a plurality of times, the sensitive layer 4 having a sufficient thickness (in other words, a sufficient contact area with the gas to be detected) can be formed. In addition, when the surface of the conductive polymer particles 41 and the polyelectrolyte 42 are attracted by electrostatic attraction, a set of the first step S10 and the second step S20 are repeated a plurality of times, and thus the charge density of the surface increases, and it is possible to obtain the highly sensitive layer 4 that more easily adsorbs a gas having a positive charge or a gas having a negative charge.

When a set of the first step S10 and the second step S20 is performed a plurality of times, the number of times can be, for example, 1 to 70, and is preferably 5 to 60.

For example, when the conductive polymer particles 41 contain an emeraldine salt of polyaniline represented by Formula (1) and the polyelectrolyte 42 forms the sensitive layer 4 containing polystyrene sulfonic acid, the above set is performed 50 times, and thus the sensitive layer 4 having a thickness of about 50 μm is obtained.

The surface porosity of the sensitive layer 4 can be adjusted by controlling the amount of the conductive polymer particles 41 adhered to the quartz crystal unit 1 by changing the amount of the first solution applied using a method of changing the immersion time for which the quartz crystal unit 1 is immersed in the first solution, for example, in the sensitive layer forming step. Specifically, the surface porosity of the sensitive layer 4 can be increased by increasing the immersion time and increasing the amount of the first solution applied.

The density of the sensitive layer 4 can be adjusted by changing the particle size of the conductive polymer particles 41 dispersed in the first solution, for example, in the sensitive layer forming step. Specifically, the density of the sensitive layer 4 can be lowered by increasing the particle size of the conductive polymer particles 41.

Next, the protective mask 5 is removed from the quartz crystal unit 1 with the sensitive layer 4 formed (S6). The protective mask 5 can be removed by a known method according to the material of the protective mask 5 used. For example, when a thermal release resin sheet is used as the protective mask 5, the protective mask 5 can be removed by a method of performing heating at a predetermined temperature for a predetermined time. In addition, for example, when a mask made of a thermosetting resin or a photocurable resin is used as the protective mask 5, the protective mask 5 can be removed by a method of performing dissolution in an organic solvent or the like.

According to the above steps, the gas sensor 10 of the present embodiment is obtained.

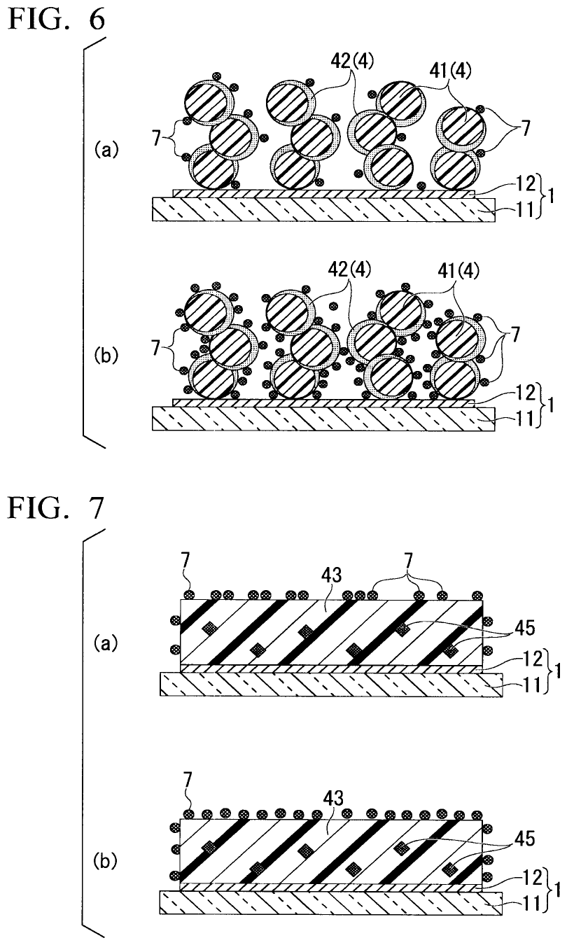

Here, the function of the gas sensor 10 of the present embodiment will be described with reference to the drawings. FIG. 6 show illustrative diagrams for illustrating effects of the gas sensor according to the present embodiment. FIG. 7 is a schematic view for illustrating an example of a gas sensor in the related art. In FIG. 6 and FIG. 7, Reference Numeral 11 indicates a crystal plate, Reference Numeral 12 indicates an electrode, and Reference Numeral 1 indicates the quartz crystal unit 1. In addition, in FIG. 6 and FIG. 7, Reference Numeral 7 indicates a gas to be detected.

As shown in FIG. 6, the gas sensor 10 of the present embodiment has the porous sensitive layer 4 that adsorbs a gas to be detected 7 and has a particle, skeleton made of a plurality of the conductive polymer particles 41 and the polyelectrolyte 42 that is at least partially disposed between the adjacent conductive polymer particles 41.

On the other hand, the sensitive film of the gas sensor in the related art shown in FIG. 7 is obtained by a method in which a resin composition in which a resin forming a coating film and a dopant 45 are dissolved in a solvent is applied onto the quartz crystal unit 1 and dried, and is a dense film 43 containing the pore-free dopant 45.

In the gas sensor 10 of the present embodiment shown in FIG. 6, the sensitive layer 4 is porous. Therefore, in the sensitive layer 4 of the gas sensor 10, compared to a non-porous sensitive film composed of the dense film 43 containing the dopant 45, when the installation areas of the sensitive layer 4 and the sensitive film are the same, the contact area with the gas to be detected 7 becomes large. Therefore, as shown in FIG. 6(a), the gas sensor 10 of the present embodiment can detect a low concentration of the gas to be detected 7 and has high sensitivity. Moreover, as shown in FIG. 6(b), even if the gas to be detected 7 has a high concentration, since the amount of a gas 7 that can be adsorbed to the sensitive layer 4 is large, the range of the detectable gas concentration is wide. Therefore, the gas sensor 10 of the present embodiment can be made small.

On the other hand, in the gas sensor in the related art shown in FIG. 7, since the sensitive film is the dense film 43, the gas to be detected 7 is unlikely to enter the film 43. Therefore, the dopant 45 and the gas to be detected 7 are unlikely to come into contact with each other and it is difficult to obtain an effect of improving the sensitivity according to the inclusion of the dopant 45. As a result, as shown in FIG. 7(a), when the gas to be detected 7 has a low concentration, sufficient sensitivity is not obtained.

In addition since the gas sensor in the related art shown in FIG. 7 is the dense film 43, the gas 7 can be adsorbed only on the outer surface of the film 43, and the amount of the gas 7 that can be adsorbed is small. As a result, as shown in FIG. 7(b), when the gas to be detected 7 has a high concentration, it is difficult to obtain the amount of change in the frequency of the gas sensor corresponding to the concentration of the gas to be detected 7 and the sensitivity is lowered. Therefore, when the gas sensor in the related art shown in FIG. 7 is used, it is difficult to widen the detectable gas concentration range without widening the installation area.

The method of producing the gas sensor 10 of the present embodiment includes a sensitive layer forming step in which the porous sensitive layer 4 that adsorbs a gas to be detected is formed on the quartz crystal unit 1. Then, the sensitive layer forming step includes the first step S10 in which the first solution in which the conductive polymer particles 41 are dispersed on the sensitive layer forming surface of the quartz crystal unit 1 is applied and dried and the second step S20 in which the second solution in which the polyelectrolyte 42 is dissolved is applied to the sensitive layer forming surface after the first step S10 and dried. Therefore, according to the method of producing the gas sensor 10 of the present embodiment, the gas sensor 10 of the present embodiment can be produced.

The gas sensor 10 of the present embodiment can be preferably used, for example, when the gas to be detected 7 is carbon dioxide, ethanol, acetone, formaldehyde, methane, ammonia or the like.

In the gas sensor 10 of the present embodiment, a case including the buffer layer 3 will be exemplified, but the buffer layer 3 may be provided as necessary or may not be provided. When a gas sensor without the buffer layer 3 is produced, the sensitive layer forming step is performed without performing the buffer layer forming step, and the porous sensitive layer 4 may be formed on the central area 12a (sensitive layer forming surface) of the electrode 12 in the quartz crystal unit 1 with the protective mask 5 formed.

### “Gas Measuring Apparatus”

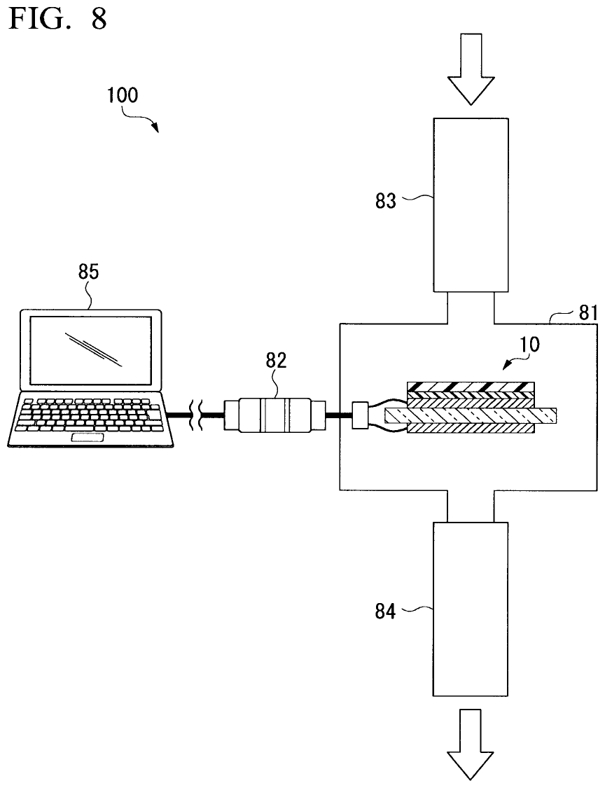

Next, a gas measuring apparatus of the present embodiment will be exemplified. FIG. 8 is a schematic view showing the gas measuring apparatus of the present embodiment. A gas measuring apparatus 100 of the present embodiment includes the gas sensor 10 of the present embodiment, a flow cell 81, a gas supply unit 83, a gas discharge unit 84, a frequency measuring apparatus 82, and a personal computer 85.

The flow cell 81 accommodates the gas sensor 10. In the gas measuring apparatus 100 of the present embodiment, the gas sensor 10 and the gas to be detected are brought into contact with each other in the flow cell 81, and thus the concentration of the gas to be detected is measured.

The gas supply unit 83 supplies a gas to be detected and a base gas to the flow cell 81 at a predetermined mixing ratio and a flow rate.

The gas discharge unit 84 discharges the gas to be detected and the base gas from the flow cell 81.

The frequency measuring apparatus 82 measures the oscillation vibration (frequency) of the quartz crystal unit 1 and detects the amount of change in the frequency. The frequency measuring apparatus 82 is electrically connected to the connection areas 12b of the two electrodes 12 of the gas sensor 10 by lead wires.

The personal computer 85 is connected to the frequency measuring apparatus 82 in a wired or wireless communication manner, and outputs the results detected by the frequency measuring apparatus 82.

In the gas measuring apparatus 100 of the present embodiment, for members other than the gas sensor 10, known members can be used.

Since the gas measuring apparatus 100 of the present embodiment includes the gas sensor 10 of the present embodiment, it has high sensitivity and the detectable gas concentration range is wide.

The embodiments of the present disclosure have been described above in detail, and configurations and combinations thereof in the embodiments are only examples, and additions, omissions, substitutions and other modifications of the configurations can be made without departing from the scope and spirit of the present disclosure.

### EXAMPLES

### “Production of Gas Sensor 1”

A gas sensor 1 was produced by the following method. First, as shown in FIG. 5(a), the quartz crystal unit 1 (product name; SEN-9E-H-10, a fundamental frequency of 9 MHz, commercially available from Tamadevice Co., Ltd.) including the circular crystal plate 11 having a diameter of 8.7 mm in a plan view and the electrodes 12 provided on both surfaces of the crystal plate 11 and of which the central area 12a was composed of a gold film having a diameter of 5.0 mm was prepared.

Next, in the quartz crystal unit 1, both surfaces of the quartz crystal unit 1 were washed by a method of performing immersion in in acetone and ultrasonical washing for 5 minutes and then immersion in pure water and ultrasonical washing for 5 minutes, and then drying at a temperature of 70° C. in the atmosphere (S1).

Next, a protective mask was formed on the surface of the quartz crystal unit 1 (S2). As shown in FIG. 5(b) and FIG. 5(c), the protective mask 5 was formed on one surface of the quartz crystal unit 1 to cover the surface of the quartz crystal unit 1 by exposing only the central area 12a (sensitive layer forming surface) of the electrode 12. The protective mask 5 was also formed on the entire other surface of the quartz crystal unit 1. As the protective mask 5, a thermal release resin sheet (product name; Revalpha (registered trademark), commercially available from Nitto Denko Corporation) was used.

### (Buffer Layer Forming Step)

Next, as shown in FIG. 5(d), the buffer layer 3 was formed on the central area 12a (sensitive layer forming surface) of the electrode 12 in the quartz crystal unit 1 with the protective mask 5 formed. As the buffer layer 3, a layer having a 3-layer structure in which the first buffer layer 31a, the second buffer layer 32 and the third buffer layer 31b were laminated in order from the side of the quartz crystal unit 1 was formed.

First, as a buffer solution for the first buffer layer 31a, a 10 mass % solution in which polystyrene sulfonic acid was dissolved in water was prepared.

Next, the quartz crystal unit 1 with the protective mask 5 formed was immersed in a buffer solution, and the buffer solution was applied (S3). Then, the quartz crystal unit 1 was removed from the buffer solution and washed with water (S4), and dried in a nitrogen atmosphere at 80° C. for 10 minutes (S5) to form the first buffer layer 31a.

Next, as a buffer solution for the second buffer layer 32, a 10 mass % solution in which polydiallyldimethylammonium chloride was dissolved in water was prepared. The second buffer layer 32 was formed by performing the above steps (S3) to (S5) in the same manner s in the first buffer layer 31a except that the buffer solution prepared in this manner was used.

Next, using the same buffer solution as for the first buffer layer 31a, the third buffer layer 31b was formed by performing the above steps (S3) to (S5) in the same manner as in the first buffer layer 31a.

### (Sensitive Layer Forming Step)

Next, a sensitive layer forming step in which the porous film sensitive layer 4 that adsorbs a gas to be detected was formed on the quartz crystal unit 1 with the buffer layer 3 formed was performed.

### (First Step)

First, as the conductive polymer particles 41 containing the emeraldine salt of polyaniline represented by Formula (1), particles of a polyaniline emeraldine salt (commercially available from Sigma-Aldrich) classified using a swirling airflow-driven air classifier (Aerofine Classifier; commercially available from Nisshin Engineering Inc.) and having a particle size (D50) of 0.6 μm were prepared. The particle size of the polyaniline emeraldine salt was measured using a laser diffraction particle size distribution measuring machine (Zetasizer Nano; commercially available from Malvern Instruments Ltd.). Then, a 1 mass % first solution in which the conductive polymer particles 41 were dispersed in N-methylpyrrolidone was prepared.

Next, the quartz crystal unit 1 with the buffer layer 3 formed was immersed in the first solution for 10 seconds. and the first solution was applied (S11). Then, the quartz crystal unit 1 was removed from the first solution and washed with water (S12), and vacuum-dried in a vacuum atmosphere at 80° C. for 10 minutes (S13). Thereby, as shown in FIG. 5(e), the conductive polymer particles 41 adhered to the sensitive layer forming surface of the quartz crystal unit 1 with the buffer layer 3 therebetween.

### (Second Step)

First, a 10 mass % second solution in which polystyrene sulfonic acid, which was the polyelectrolyte 42, was dissolved in water was prepared.

Next, the quartz crystal unit 1 to which the conductive polymer particles 41 adhered was immersed in a second solution, and the second solution was applied (S14). Then, the quartz crystal unit 1 was removed from the second solution and washed with water (S15) and dried in a nitrogen atmosphere at 80° C. for 10 minutes (S16). Thereby, as shown in FIG. 5(f), the polyelectrolyte 42 adhered to the conductive polymer particles 41 formed on the sensitive layer forming surface of the quartz crystal unit 1.

Then, a set of the first step S10 and the second step S20 was repeated five times, and thus the porous sensitive layer 4 having a thickness of 5 μm was formed.

Next, the protective mask 5 was removed from the quartz crystal unit 1 with the sensitive layer 4 formed (S6). The protective mask 5 was removed by a method of heating the protective mask 5 for 10 minutes at a temperature of 110° C. in a nitrogen atmosphere.

According to the above steps the gas sensor 1 is obtained.

### “Production of Gas Sensor 2”

A gas sensor 2 was obtained in the same manner as in the gas sensor 1 except that, as the conductive polymer particles 41, particles of a polyaniline emeraldine salt (commercially available from Sigma-Aldrich) classified using a swirling airflow-driven air classifier (Aerofine Classifier; commercially available from Nisshin Engineering Inc.)

and having a particle size (D50) of 2 μm were used, and the immersion time for which the quartz crystal unit 1 with the buffer layer 3 formed was immersed in the first solution was set to 20 seconds. The particle size of the polyaniline emeraldine salt was measured using a laser diffraction particle size distribution measuring machine (Zetasizer Nano; commercially available from Malvern Instruments Ltd.).

### “Production of Gas Sensor 3”

A gas sensor 3 was obtained in the same manner as in the gas sensor 1 except that, as the conductive polymer particles 41, particles of a polyaniline emeraldine salt (commercially available from Sigma-Aldrich) classified using a swirling airflow-driven air classifier (Aerofine Classifier; commercially available from Nisshin Engineering Inc.) and having a particle size (D50) of 1.7 μm were used, and the immersion time for which the quartz crystal unit 1 with the buffer layer 3 formed was immersed in the first solution was set to 30 seconds. The particle size of the polyaniline emeraldine salt was measured using a laser diffraction particle size distribution measuring machine (Zetasizer Nano; commercially available from Malvern Instruments Ltd.).

### “Production of Gas Sensor 4”

A gas sensor 4 was obtained in the same manner as in the gas sensor 1 except that, as the conductive polymer particles 41, particles of a polyaniline emeraldine salt (commercially available from Sigma-Aldrich) classified using a wirling airflow-driven air classifier (Aerofine Classifier; commercially available from Nisshin Engineering Inc.) and having a particle size (D50) of 2.0 μm were used and the immersion time for which the quartz crystal unit 1 with the buffer layer 3 formed was immersed in the first solution was set to 40 seconds. The particle size of the polyaniline emeraldine salt was measured using a laser diffraction particle size distribution measuring machine (Zetasizer Nano; commercially available from Malvern Instruments Ltd.).

### “Production of Gas Sensor 5”

A gas sensor 5 was obtained in the same manner as in the gas sensor 1 except that a 10 mass % second solution in which polyacrylic acid was dissolved in water was prepared in the second step. “Production of Gas Sensor 6”

A gas sensor 6 was obtained in the same manner as in the gas sensor 2 except that a 10 mass % second solution in which polyacrylic acid was dissolved in water was prepared in the second step.

### “Production of Gas Sensor 7”

A gas sensor 7 was obtained in the same manner as in the gas sensor 3 except that a 10 mass % second solution in which polyacrylic acid was dissolved in water was prepared in the second step.

“Production of gas sensor 8”

A gas sensor 8 was obtained in the same manner as in the gas sensor 4 except that a 10 mass % second solution in which polyacrylic acid was dissolved in water was prepared in the second step.

The surface porosity and the film density of the gas sensors 1 to 8 obtained in this manner were measured by the following methods. The results are shown in Table

### (Surface Porosity)

For each gas sensor, using a scanning electron microscope (SEM) (product name; ST58000, commercially available from Hitachi High-Tech Corporation), the sensitive layer 4 was observed at 10 locations. For the obtained SEM image, a conductive polymer particle portion and a void portion were each binarized, the ratio of the void portion to the installation area of the sensitive layer 4 was calculated, and the average value of 10 locations was used as the surface porosity.

### (Film Density)

The film density of the sensitive layer 4 formed on the quartz crystal unit 1 was measured using the Archimedes method.

Specifically, the weight of the quartz crystal unit 1 was subtracted from the quartz crystal unit 1 with the sensitive layer 4 formed, and thus the weight of the sensitive layer 4 in the atmosphere was measured. Then, the quartz crystal unit 1 with the sensitive layer 4 formed was immersed in water, the weight in water was measured, the weight of the quartz crystal unit 1 was subtracted, and the weight of the sensitive layer 4 in water was measured. Using the weight of the sensitive layer 4 in the atmosphere obtained in this manner and the weight of the sensitive layer 4 in water, the layer density (g/cm³) of the sensitive layer 4 was calculated by the following formula. The results are shown in Table 1.

ρ=P_{L}+A(P₀−P_{L})/(A−B)

(A in the formula is the weight in the atmosphere, B is the weight in water, P₀ is the density of water, and P_{L} is the density of air)

TABLE 1

| | | | | | | Amount of | |
|---|---|---|---|---|---|---|---|
| | | | Layer | | | change in | Gas |
| | Gas | Surface | density | Conductive | | frequency | concentration |
| | sensor | porosity | (g/cm³) | polymer | Electrolyte | (Hz) | (ppm) |
| Example 1 | 1 | 15 | 0.073 | Polyaniline | Polystyrene | 10 | 5 |
| | | | | | sulfonic acid | | |
| Example 2 | 1 | 15 | 0.073 | Polyaniline | Polystyrene | 18 | 10 |
| | | | | | sulfonic acid | | |
| Example 3 | 1 | 15 | 0.073 | Polyaniline | Polystyrene | 36 | 20 |
| | | | | | sulfonic acid | | |
| Example 4 | 2 | 20 | 0.056 | Polyaniline | Polystyrene | 15 | 5 |
| | | | | | sulfonic acid | | |
| Example 5 | 2 | 20 | 0.056 | Polyaniline | Polystyrene | 27 | 10 |
| | | | | | sulfonic acid | | |
| Example 6 | 2 | 20 | 0.056 | Polyaniline | Polystyrene | 43 | 20 |
| | | | | | sulfonic acid | | |
| Example 7 | 3 | 25 | 0.039 | Polyaniline | Polystyrene | 20 | 5 |
| | | | | | sulfonic acid | | |
| Example 8 | 3 | 25 | 0.039 | Polyaniline | Polystyrene | 30 | 10 |
| | | | | | sulfonic acid | | |
| Example 9 | 3 | 25 | 0.039 | Polyaniline | Polystyrene | 60 | 20 |
| | | | | | sulfonic acid | | |
| Example 10 | 4 | 30 | 0.030 | Polyaniline | Polystyrene | 25 | 5 |
| | | | | | sulfonic acid | | |
| Example 11 | 5 | 15 | 0.070 | Polyaniline | Polyacrylic | 5 | 5 |
| | | | | | acid | | |
| Example 12 | 6 | 20 | 0.082 | Polyaniline | Polyacrylic | 8 | 5 |
| | | | | | acid | | |
| Example 13 | 7 | 25 | 0.065 | Polyaniline | Polyacrylic | 14 | 5 |
| | | | | | acid | | |
| Example 14 | 8 | 30 | 0.047 | Polyaniline | Polyacrylic | 18 | 5 |
| | | | | | acid | | |
| Comparative | | 0 | 1.1 | Polyaniline | — | 2 | 5 |
| Example 1 | | | | | | | |
| Comparative | | 0 | 1.1 | Polyaniline | — | 3 | 20 |
| Example 2 | | | | | | | |

### Example 1 to Example 14

Next, as the gas sensor 10, the gas sensor shown in Table 1 was installed in the flow cell 81, the gas measuring apparatus 100 shown in FIG. 8 in which product name; THQ-100P-SW type (commercially available from Tamadevice Co., Ltd.) was installed was used as the frequency measuring apparatus 82, and the amount of change in the frequency of the gas sensor with respect to the concentration of the gas to be detected shown in Table 1 was examined by the following method.

An electric field was applied to the electrode 12 of the quartz crystal unit 1 by the frequency measuring apparatus 82 via lead wires. While the oscillation vibration (frequency) of the quartz crystal unit 1 was continuously measured by the frequency measuring apparatus 82, the gas supply unit 83 and the gas discharge unit 84 were operated, a base gas composed of dry air was supplied to the flow cell 81 at a flow rate of 200 cc/sec for 1 hour, and thus the fundamental frequency (base line) was stabilized.

Then, the gas supply unit 83 mixed a base gas and ethanol gas, which is a gas to be detected so that the gas concentration shown in Table 1 was obtained and supplied the mixed gas to the flow cell 81 at a flow rate of 200 cc/sec, and the vibration (frequency) was measured by the frequency measuring apparatus 82 for 5 minutes.

Next, the gas supply unit 83 supplied the base gas to the flow cell 81 at a flow rate of 200 cc/sec until the oscillation vibration (frequency) of the quartz crystal unit 1 reached the base line.

Then, the frequency measuring apparatus 82 detected the difference between the average value of the vibration (frequency) when ethanol gas was supplied at a gas concentration shown in Table 1 and the base line (the amount of change in the frequency), and the result was output to the personal computer 85. Table 1 shows the output amounts of change in the frequency.

### Comparative Example 1 and Comparative Example 2

### “Production of Gas Sensor”

The quartz crystal unit 1 was prepared in the same manner as in Example 1, both surfaces were washed in the same manner as in Example 1, and a protective mask was formed in the same manner as in Example 1. Then, a sensitive film having a thickness of 5 μm was formed on the central area 12a (sensitive layer forming surface) of the electrode 12 in the quartz crystal unit 1 with the protective mask 5 formed.

The sensitive film was formed by a method in which a 10 mass % solution obtained by dissolving a polyaniline emeraldine salt (commercially available from Sigma-Aldrich) in tetramethyluric acid was applied to the central area 12a of the electrode 12 by a drop casting method and dried in a nitrogen atmosphere at 110° C. for 30 minutes.

Next, the protective mask 5 was removed in the same manner as in Example 1 from the quartz crystal unit 1 with the sensitive film formed.

According to the above steps gas sensors of comparative examples were obtained.

The amount of change in the frequency of the gas sensor with respect to the concentration of the gas to be detected shown in Table 1 was examined using the gas measuring apparatus 100 in the same manner as in Example 1 except that the gas sensor of the comparative example obtained in this manner was installed in the flow cell 81. The results are shown in Table 1.

As shown in Table 1, when the gas concentration of ethanol gas was a low concentration of 5 ppm, the amount of change in the frequency, of Examples 1, 4, 7, and 10 to 14 using the gas sensors 1 to 8 was sufficiently larger than that of Comparative Example 1 using the gas sensor of the comparative example, and the sensitivity was high.

In addition, when the gas concentration of ethanol gas was a high concentration of 20 ppm, the amount of change in the frequency of Examples 3, 6, and 9 using the gas sensors 1 to 3 was sufficiently larger than Comparative Example 2 using the gas sensor of the comparative example, and the sensitivity was high. In addition, according to the amount of change in the frequency of Examples 1 to 9 using the gas sensors 1 to 3, in the gas sensors 1 to 3, when the gas to be detected concentration was a higher concentration, the amount of change in the frequency of the gas sensor was large. Accordingly, it was confirmed that the gas sensors 1 to 3 had a wide detectable gas concentration range.

On the other hand, this was speculated to be because, in the gas sensor of the comparative example, since the amount of the gas that could be adsorbed was small, even if the gas concentration was a high concentration, the amount of change in the frequency was not sufficiently large, resulting in poor sensitivity.

In addition, as shown in Table 1, when the gas concentration of ethanol gas was a low concentration of 5 ppm, the amount of change in the frequency of Examples 1, 4, 7, and 10 using the gas sensors 1 to 4 was larger as the surface porosity was larger. This was speculated to be because, when the surface porosity was in a range of 30% or less, as the surface porosity was larger, the contact area between the sensitive layer and the gas to be detected was wider.

In addition, as shown in Table 1, the amount of change in the frequency of Examples 1, 4, 7, and 10 using the gas sensors 1 to 4 containing polystyrene sulfonic acid as a polyelectrolyte was larger than the amount of change in the frequency of Examples 11 to 14 using the gas sensors 5 to 8 containing polyacrylic acid as a polyelectrolyte, and the sensitivity was high.

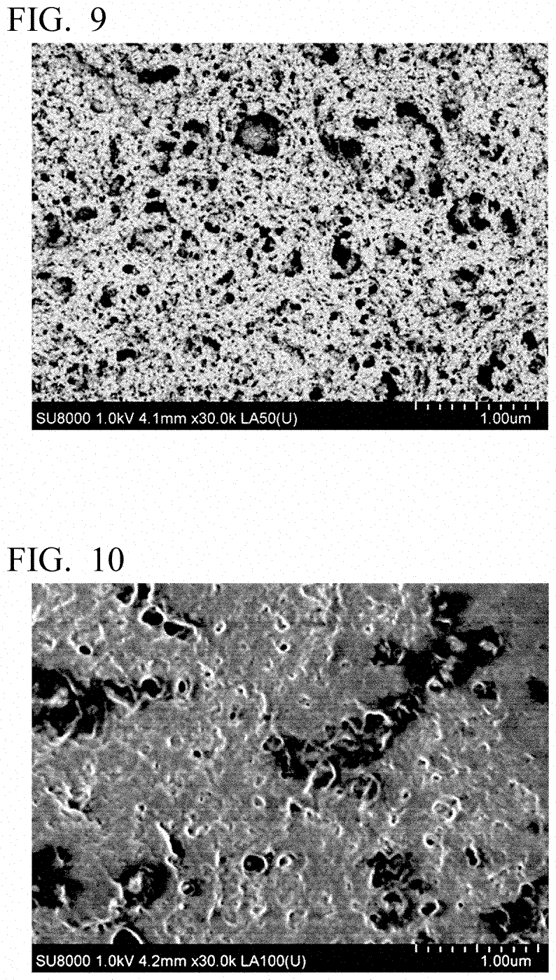

Next, the sensitive layers of the gas sensor 1 and the gas sensor of the comparative example were observed using a scanning electron microscope (SEM) (product name; SU8000, commercially available from Hitachi High-Tech Corporation). FIG. 9 is a microscopic image of the sensitive layer of the gas sensor 1. FIG. 10 is a microscopic image of the sensitive film of the gas sensor of the comparative example.

As shown in FIG. 9, the sensitive layer of the gas sensor 1 was a porous layer having a large number of pores. On the other hand, as shown in FIG. 10, the sensitive film of the gas sensor of the comparative example was not porous but dense without pores.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

### EXPLANATION OF REFERENCES

1 Quartz crystal unit
3 Buffer layer
4 Sensitive layer
5 Protective mask
5, 7 Gas
10 Gas sensor
11 Crystal plate
12 Electrode
12a Central area
12b Connection area
31a First buffer layer
31b Third buffer layer
32 Second buffer layer
41 Conductive polymer particle
42 Polyelectrolyte
43 Film
45 Dopant
81 Flow cell
82 Frequency measuring apparatus
83 Gas supply unit
84 Gas discharge unit
85 Personal computer
100 Gas measuring apparatus

1. A gas sensor comprising:
a quartz crystal unit; and
a sensitive layer formed on the quartz crystal unit and configured to adsorb a gas to be detected, wherein
the sensitive layer is a porous film and has a particle skeleton made of a plurality of conductive polymer particles and a polyelectrolyte that is at least partially disposed between adjacent conductive polymer particles.

2. The gas sensor according to claim 1, wherein a porosity of the sensitive layer on a surface is 15% to 30%.

3. The gas sensor according to claim 1, wherein a density of the sensitive layer is 0.03 g/cm³ to 0.1 g/cm³.

4. The gas sensor according to claim 1, wherein the conductive polymer particles contains a polyaniline represented by the following formula (1), wherein n is a degree of polymerization.

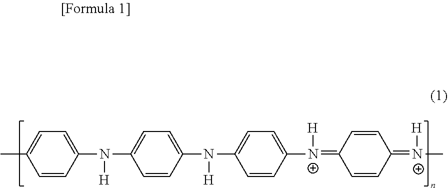

5. The gas sensor according to claim 4, wherein the polyelectrolyte contains one or more of: polystyrene sulfonic acid; polyacrylic acid; and polyvinyl phosphonic acid.

6. The gas sensor according to claim 5, wherein
a buffer layer is disposed between the quartz crystal unit and the sensitive layer, the buffer layer being in contact with the sensitive layer, and
an electrostatic adsorption layer is formed on a surface of the buffer layer in contact with the sensitive layer, the electrostatic adsorption layer containing one or more of: polystyrene sulfonic acid; polyacrylic acid; and polyvinyl phosphonic acid.

7. The gas sensor according to claim 1, wherein
the quartz crystal unit comprises a crystal plate and an electrode made of a metal film disposed on the crystal plate,
the sensitive layer is laminated on the electrode via a buffer layer,
the electrode and the conductive polymer particles have a positive charge,
the polyelectrolyte and surfaces of the buffer layer, one of which is in contact with the electrode, and other of which is in contact with the sensitive layer, have a negative charge.

8. A method of producing a gas sensor comprising a sensitive layer forming step of forming a sensitive layer on a quartz crystal unit, the sensitive layer being made of a sensitive layer, which is porous and configured to adsorb a gas to be detected, wherein
the sensitive layer forming step comprises:
a first step of applying a first solution, in which conductive polymer particles are dispersed, on a surface for forming a sensitive layer on the quartz crystal unit and drying an applied first solution; and
a second step of applying a second solution, in which polyelectrolyte is dissolved, on the surface for forming a sensitive layer after the first step and drying the applied second solution.

9. The method of producing a gas sensor according to claim 8, wherein the conductive polymer particles include an emeraldine salt of polyaniline represented by a formula (1), wherein n is a degree of polymerization.

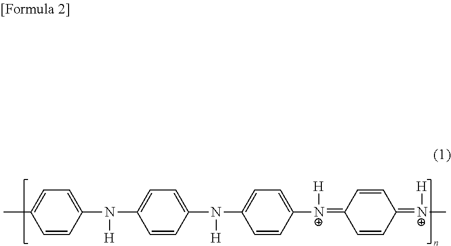

10. The method of producing a gas sensor according to claim 9, wherein the polyelectrolyte contains one or more of: polystyrene sulfonic acid; polyacrylic acid; and polyvinyl phosphonic acid.

11. The method of producing a gas sensor according to claim 9, the method further comprising a buffer layer forming step of forming a buffer layer including a an electrostatic adsorption layer containing one or more of: polystyrene sulfonic acid; polyacrylic acid; and polyvinyl phosphonic acid on the surface for forming a sensitive layer, wherein
the sensitive layer in contact with the electrostatic adsorption layer is formed in the sensitive layer forming step.

12. The method of producing a gas sensor according to claim 8, wherein
the quartz crystal unit comprises a crystal plate and an electrode made of a metal film disposed on the crystal plate,
the method further comprises a buffer layer forming step of forming a buffer layer on the electrode, surfaces of the buffer layer in contact with the electrode and the sensitive layer having a negative charge,
in the first step, a first solution, in which conductive polymer particles having a positive charge are dispersed, is used, and
in the second step, a second solution, in which polyelectrolyte having a negative charge is dissolved, is used.

13. The method of producing a gas sensor according to claim 8, wherein a set of the first step and the second step is performed multiple times in the sensitive layer forming step.

14. A gas measuring apparatus comprising the gas sensor according to claim 1.
